SEQUENCE LISTING (9 sequences)

SEQ ID NO: 1 - DNA, 901 bp, Homo sapiens
tccggagcccgccccgcccccgagccgtagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcattggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 2 - protein, 301 aa, Homo sapiens
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 3 - protein, 20 aa, Homo sapiens
AMVEVQLDADHDYPFLAEVV

SEQ ID NO: 4 - protein, 20 aa, Homo sapiens
AMVEVQLETQYQYPFLAEVV

SEQ ID NO: 5 - protein, 20 aa, Homo sapiens
AMVEVQLESDHEYPFLAEVV

SEQ ID NO: 6 - protein, 20 aa, Drosophila melanogaster
AMVEVQLDHDTNVPFLLEIA

SEQ ID NO: 7 - protein, 20 aa, Mus musculus
AMVEVQLDTDHDYPFLAEVV

SEQ ID NO: 8 - protein, 20 aa, Rattus norvegicus
AMVEVQLDTDHDYPFLAEVV

SEQ ID NO: 9 - protein, 20 aa, Gallus gallus
AMVEVQLDAEHDYPFLAEVV

(19) United States
(12) Patent Application Publication
(10) Pub. No.: US 2008/0096227 A1
(43) Pub. Date: Apr. 24, 2008

(54) CRAC modulators and use of same for drug discovery

(76) Inventors: Reinhold Penner, Honolulu, HI (US); Andrea Fleig, Honolulu, HI (US); Jean-Pierre Kinet, Boston, MA (US)

Correspondence Address: MORGAN, LEWIS & BOCKIUS, LLP, ONE MARKET SPEAR STREET TOWER, SAN FRANCISCO, CA, 94105, US

(21) Appl. No.: 11/733,640

(22) Filed: Apr. 10, 2007

Related U.S. Application Data
(60) Provisional application No. 60/791,038, filed on Apr. 10, 2006.

(51) Int. Cl. C12N 5/02; G01N 33/53

(52) U.S. Cl. 435/7.2; 435/375

(57) ABSTRACT

The invention relates to use of a calcium release activated Ca⁺² (CRAC) channel (CRACM) such as CRACM1 and CRACM2 to identify bioactive agents which can modulate store operated calcium entry and CRAC channel activity. The invention further relates to the use of recombinant nucleic acids that encode CRACM. One aspect of the invention includes methods of determining binding of candidate bioactive agents to a CRACM polypeptide and for determining modulation of CRACM polypeptide activity as it affects CRAC channel permeability. The invention further relates to methods and compositions modulating the cellular expression of the nucleic acids that encode CRACM.

### CROSS REFERENCE TO RELATED APPLICATIONS

This application claims the benefit under 35 U.S.C. §119(e) to provisional application 60/791,038, filed Apr. 10, 2006, herein incorporated by reference.

### STATEMENT REGARDING FEDERALLY SPONSORED RESEARCH OR DEVELOPMENT

This work was supported in part by NIH grants 5-R37-GM053950 (JPK), R01-AI050200 and R01-NS040927 (RP), R01-GM065360 (AF).

### BACKGROUND OF THE INVENTION

Receptor-mediated signaling in non-excitable cells, immune cells in particular, involves an initial rise in intracellular Ca²⁺ due to release from the intracellular stores. The resulting depletion of the intracellular stores induces Ca² entry through the plasma membrane via calcium release-activated calcium (CRAC) channels (J. W. Putney, Jr., Cell Calcium 11, 611 (November-December, 1990); M. Hoth, R. Penner, Nature 355, 353 (Jan. 23, 1992); A. B. Parekh, R. Penner, Physiol Rev 77, 901 (1997)). This phenomenon is central to many physiological processes such as gene transcription, proliferation and cytokine release (A. B. Parekh, R. Penner, Physiol Rev 77, 901 (1997); M. Partiseti et al., J Biol Chem 269, 32327 (Dec. 23, 1994); R. S. Lewis, Annu Rev Immunol 19, 497 (2001)). Biophysically, CRAC currents have been well characterized (M. Hoth, R. Penner, Nature 355, 353 (Jan. 23, 1992); M. Hoth, R. Penner, J Physiol (Lond) 465, 359 (1993); A. Zweifach, R. S. Lewis, Proc Natl Acad Sci USA 90, 6295 (1993)), but the identity of the CRAC channel itself and the pathway resulting in its activation are still unknown. Recently, two groups independently identified STIM1 to be an essential component of the store-operated calcium entry (J. Liou et al., Curr Biol 15, 1235 (Jul. 12, 2005); J. Roos et al., J Cell Biol 169, 435 (May 9, 2005)). This protein is located in intracellular compartments that likely represent parts of the ER. It has a single transmembrane spanning domain with a luminal EF-hand motif that appears to be crucial for its hypothesized function as the ER sensor for luminal Ca²⁺ levels. Upon store depletion, STIM1 redistributes into distinct structures (punctae) that move and accumulate underneath the plasma membrane. Whether or not STIM1 actually incorporates into the plasma membrane is controversial (J. Liou et al., Curr Biol 15, 1235 (Jul. 12, 2005); S. L. Zhang et al., Nature 437, 902 (Oct. 6, 2005); M. A. Spassova et al., Proc Natl Acad Sci USA 103, 4040 (Mar. 14, 2006)). STIM1 is required to activate CRAC currents, however, its presence or even its translocation is not sufficient, since lymphocytes from SCID patients have normal STIM1 levels, yet fail to activate CRAC channels (S. Feske, et al., J Exp Med 202, 651 (Sep. 5, 2005)). This suggests that other molecular components participate in the store-operated Ca²⁺ entry mechanism.

### SUMMARY OF THE INVENTION

The invention relates to use of a calcium release activated Ca⁺² (CRAC) channel modulators (CRACM) such as CRACM1 and CRACM2. The invention further relates to the use of recombinant nucleic acids that encode CRACM. One aspect of the invention includes methods of determining whether candidate bioactive agents are able to modulate the ion channel activity of a CRACM polypeptide. Also encompassed by the invention are methods of screening for agents that are able to modulate CRACM polypeptide activity as it affects CRAC channel permeability. The invention further relates to methods and compositions modulating the cellular expression of the nucleic acids that encode CRACM.

One aspect of the invention provides methods for screening for candidate bioactive agents that bind to a CRACM polypeptide. In this method, a CRACM polypeptide is contacted with a candidate agent, and it is determined whether the candidate agent binds to the CRACM polypeptide. An embodiment of the invention provides for contacting a CRACM polypeptide with a library of two or more candidate agents and then determining the binding of one or more of the candidate agents to CRACM polypeptide. In a preferred embodiment, the CRACM polypeptide comprises CRACM1 having the amino acid sequence as set forth in FIG. 4 or the Drosophila CRACM2 polypeptide.

In a further embodiment, the invention provides methods for screening for bioactive candidate agents that modulate the CRAC activity of a cell. In this embodiment, the cell is contacted with a candidate agent, and the modulation of the divalent cation permeability is detected. In some embodiments, the candidate agent(s) increase the cation permeability. In other embodiments, the candidate agent(s) decrease the cation permeability. The preferred cation is Ca⁺²

It is further an object of the invention to provide methods for screening for candidate bioactive agents that are capable of modulating expression of the CRACM polypeptide. In this method, a recombinant cell is provided which is capable of expressing a CRACM polypeptide. The recombinant cell is contacted with a candidate agent, and the effect of the candidate agent on CRACM polypeptide expression is determined. In some embodiments, the candidate agent may comprise a small molecule, protein, polypeptide, or nucleic acid (e.g., antisense nucleic acid). In another embodiment of the invention, CRACM polypeptide expression levels are determined in the presence of a candidate agent and these levels are compared to endogenous CRACM expression levels. Those candidate agents which regulate CRACM polypeptide expression can be tested in non-recombinant cells to determine if the same effect is reproduced.

The invention also provides a method for inhibiting CRAC activity comprising contacting at least one cell with (1) an agent that inhibits CRACM expression and/or an agent that inhibits a CRACM polypeptide.

Antisense CRACM nucleic acids as well as anti-CRACM antibodies are also encompassed by the invention.

### BRIEF DESCRIPTION OF THE DRAWINGS

FIG. 1 depicts identification of CRACM1 and CRACM2 as crucial regulators of store-operated Ca²⁺ entry in Drosophila. Ca²⁺ signals measured in Drosophila S2R+ cells in the primary high-throughput screen using an automated fluorometric imaging plate reader (FLIPR). (A) Fluo-4-AM fluorescence changes in relative fluorescence units (r.f.u.) obtained from CRACM1 dsRNA. Reference traces are provided for Rho1 dsRNA (mock) and STIM1 dsRNA. Cells were kept in Ca²⁺-free solution and exposed to thapsigargin (2 μM), followed by addition of 2 mM Ca²⁺. The traces are representative of two independent repeats of the primary screen. (B) Same protocol as in (A) but for cells treated with CRACM2 dsRNA. (C) Normalized average time course of IP₃-induced (20 μM) I_{CRAC} measured in Drosophila Kc cells. Currents of individual cells were measured at −80 mV, normalized by their respective cell size, averaged and plotted versus time (±S.E.M.). Cytosolic calcium was clamped to 150 nM using 10 mM BAPTA and 4 mM CaCl₂. Traces correspond to untreated control (wt; black closed circles, n=10), Rho1 dsRNA (mock; open circles, n=8), CRACM1 dsRNA (red circles, n=6) and CRACM2 dsRNA (green circles, n=9). (D) Leak-subtracted, normalized and averaged current-voltage (I/V) data traces of I_{CRAC} extracted from representative cells at 60 s from currents evoked by 50 ms voltage ramps from −100 to +100 mV. Traces correspond to untreated control (wt, n=9), CRACM1 dsRNA (n=5) and CRACM2 dsRNA (n=6). (E) Same as panel (C), except that IP₃ was omitted and [Ca²⁺]i was clamped close to zero by 10 mM BAPTA to induce passive store depletion. Traces correspond to untreated control (wt; black circles, n=4) and CRACM1 dsRNA (red circles, n=3). (F) Leak-subtracted, normalized and averaged current-voltage (I/V) data traces of I_{CRAC} extracted from representative cells at 200 s from currents evoked by 50 ms voltage ramps from −100 to +100 mV. Traces correspond to passive depletion-induced I_{CRAC} obtained from untreated control (wt, n=4) and CRACM1 dsRNA (n=3).

FIG. 2 depicts suppression of store-operated Ca²⁺ entry and I_{CRAC} by CRACM1 siRNA in HEK293 and Jurkat cells. (A) Left panel: Reverse transcription-polymerase chain reaction (RT-PCR) of CRACM1 mRNA from HEK293 cells infected with two different CRACM1-specific siRNAs and a scrambled sequence control. Number of cycles: 24, 27, 30. Right panel: Positive control for RT-PCR used primers specific for small ribosomal protein. Number of cycles: 24, 27, 30. (B) Fura-2-AM fluorescence measurements of [Ca²⁺]i in cells treated with scramble (control) and two different CRACM1-specific siRNAs in HEK293 cells. Cells were kept in Ca²⁺-free solution and exposed to thapsigargin (2 μM), followed by addition of 2 mM Ca²⁺. The traces are representative of three independent experiments. (C) Same protocol as in (B), but for Jurkat cells. The traces are averages of three independent experiments. (D) Normalized average time course of IP₃-induced (20 μM) I_{CRAC} measured in HEK293 cells. Traces correspond to scramble (black circles, n=13), CRACM1 siRNA-1 (red circles, n=10) and CRACM1 siRNA-2 (blue circles, n=9). (E) Leak-subtracted, normalized and averaged current-voltage (I/V) data traces of I_{CRAC} extracted from representative cells at 60 s from currents evoked by 50 ms voltage ramps from −100 to +100 mV. Traces correspond to scramble (n=10), CRACM1 siRNA-1 (n=8) and CRACM1 siRNA-2 (n=7). (F) Same as panel (D), but for Jurkat cells. Traces correspond to scramble (black circles, n=9), CRACM1 siRNA-1 (red circles, n=8) and CRACM1 siRNA-2 (blue circles, n=8). (G) Leak-subtracted, normalized and averaged current-voltage (I/V) data traces of I_{CRAC} extracted from representative cells at 60 s from currents evoked by 50 ms voltage ramps from −100 to +100 mV. Traces correspond to scramble (n=9), CRACM1 siRNA-1 (n=7) and CRACM1 siRNA-2 (n=8).

FIG. 3 depicts overexpression of CRACM1 in HEK293, Jurkat cells and RBL-2H3 cells. (A) Analysis of HEK293 cells for overexpression of CRACM1 by immunoprecipitation with anti-myc or anti-His C-term antibodies and immunoblotting with anti-myc antibody. Control immunoprecipitation from empty vector-transfected cells did not show any bands. (B) Normalized average time course of IP₃-induced (20 μM) I_{CRAC} measured in HEK293 cells. Currents of individual cells were measured at −80 mV, normalized by their respective cell size, averaged and plotted versus time (±S.E.M.). Cytosolic calcium was clamped to 150 nM using 10 mM BAPTA and 4 mM CaCl₂. Traces correspond to empty vector-transfected (control; black circles, n=13) and cells transfected with GFP plus CRACM1 (red circles, n=14). (C) Same as panel (B), but for Jurkat cells. Traces correspond to empty vector-transfected (control; black circles, n=4) and cells transfected with GFP plus CRACM1 (red circles, n=5). (D) Same as panel (B), but for RBL-2H3 cells. Traces correspond to empty vector-transfected (control; black circles, n=9) and cells transfected with GFP plus CRACM1 (red circles, n=9). (E) Immunofluorescence localization of CRACM1 in HEK293 cells visualized by confocal microscopy. Immunostaining for CRACM1-flag-N-term (upper panels) or CRACM1-myc-C-term (lower panels) in intact (left panels) and permeabilized cells (right panels). (F) Same as bottom right panel of (E), but at higher magnification of selected cells to illustrate plasma membrane staining.

FIG. 4A is the nucleic acid sequence of human CRACM1 (SEQ ID NO:1).

FIG. 4B is the amino acid sequence of human CRACM1 (SEQ ID NO:2).

FIG. 5 illustrates data from CRACM1 expressed in HEK-293 cells. (A) Co-immunoprecipitate of CRACM1 from HEK293 cells co-transfected with Flag-CRACM1 and CRACM1-Myc-His. Lane 2 shows that Flag-CRACM1 co-immunoprecipitates CRACM1-Myc-His. Lane 3 shows the reverse co-IP and Lanes 1 and 4 show the control IPs. (B) Co-immunoprecipitation of Flag-CRACM1 and Stim1-Myc-His, co-transfected in HEK-293 cells. Whole cell lysates were either immunoprecipitated with anti-myc antibody (first lane) or anti-flag antibody (second lane) and blotted with either anti-myc antibody (upper panels) or anti-flag antibody (lower panels). (C) Sequence alignment of human CRACM1 (SEQ ID NO:3), CRACM2 (SEQ ID NO:4), and CRACM3 (SEQ ID NO:5) as well as CRACM1 from various species (Drosophila (SEQ ID NO:6), mouse (SEQ ID NO:7), rat (SEQ ID NO:8), and chicken (SEQ ID NO:9)), highlighting the acidic residues and the (residue numbers pertain to the human sequence of CRACM1). (D) Co-IP of D110/112A-CRACM1 and E106Q-CRACM1 mutant with the wt-CRACM1. Lane 1 shows that D110/112A-CRACM1-Myc-His can co-IP Flag-CRACM1 and lane 3 shows that CRACM1-Myc-His can co-IP Flag-E106Q-CRACM1. Lanes 2 and 4 show the controls. (E) Confocal images of HEK293 cells transfected with Flag-CRACM1, D110/112A-CRACM1-Myc-His, Flag-E190Q-CRACM1 and Flag-E106Q-CRACM1 and stained with anti-myc or anti-flag antibodies respectively to show cellular localization of the mutants.

FIG. 6 shows the results of selectivity experiments with CRACM1 mutants. (A) Normalized average time course of IP₃-induced (20 μM) CRAC currents measured in HEK293 cells co-overexpressing STIM1 and wild-type CRACM1 (black circles, n=14) and E106Q mutation (red circles, n=9). Currents of individual cells were measured at −80 mV, normalized by cell capacitance, averaged and plotted versus time (±S.E.M.). Cytosolic calcium was clamped to near zero with 20 mM BAPTA. The bar indicates application of divalent-free (DVF) solution. (B) Average current-voltage (I/V) relationships of CRAC currents extracted from representative HEK293 cells shown in panel A at 120 s in to the experiment. Data represent leak-subtracted currents evoked by 50 ms voltage ramps from −100 to +150 mV, normalized to cell capacitance (pF). Traces correspond to STIM1+wt-CRACM1 (wt, n=12) or STIM1+E106Q mutant (n=6). (C) Normalized average time course of IP₃-induced (20 μM) currents at −80 and +130 mV produced by the E106D mutant. Cells were exposed to nominally Ca²⁺-free external solution (black circles, n=6) or Na⁺-free solution (red circles, n=6) for the time indicated by the black bar. Currents were analyzed as in panel A. (D) Average I/V traces of the E106D mutant extracted at 120 s (black trace, n=6) and at the end of the application of Ca²⁺-free (blue trace, n=6) or Na⁺-free (red trace, n=6) solutions (same cells as in panel C). Data analysis as in panel B. (E) Normalized average time course of CRAC currents in HEK293 expressing wt-CRACM1 (black circles, n=9) or E106D mutant (red circles, n=7). Analysis as in panel A. Cells were superfused with external solution containing 10 mM Ba²⁺ (and 0 Ca²⁺) at the time indicated by the black bar. Note that cells were superfused with Ba²⁺ in the absence of extracellular Na⁺ (replaced by TEA⁺) to avoid Na⁺ current contamination. (F) Average I/V data traces of currents extracted from representative HEK293 cells expressing the E106D mutant shown in panel E, before (120 s, n=4) and at the end of Ba²⁺ application (180 s, n=4). Analysis as in panel B. (G) Normalized average time course of IP₃-induced (20 μM) currents at −80 and +130 mV produced by the E190Q mutant. Cells were exposed to nominally Ca²⁺-free external solution (black circles, n=7) or Na⁺-free solution, where Ca²⁺ was substituted with Ba²⁺ (red circles, n=8) for the time indicated by the bar. Currents were analyzed as in panel A. (H) Average I/V traces of the E1900 mutant extracted at 120 s (black trace, n=8) and at the end of the application of 10 Ba²⁺ (red trace, n=8) or Ca²⁺-free solutions (blue trace, n=7; same cells as in panel G). Data analysis as in panel B.

FIG. 7 illustrates selectivity experiments with pore mutants of CRACM1. (A) Normalized average time course of IP₃-induced (20 μM) CRAC currents measured in HEK293 cells co-expressing STIM1 with either wt-CRACM1 (black circles, n=12) or the D110/112A mutant of CRACM1 (red circles, n=11). Currents of individual cells were measured at −80 mV and +130 mV, normalized by cell capacitance, averaged and plotted versus time (±S.E.M.). Cytosolic calcium was clamped to near zero with 20 mM BAPTA. The black bar indicates application of an external solution containing 10 mM Ca²⁺ with Na⁺ replaced by TEA⁺. (B) Average time course of IP₃-induced (20 μM) currents produced by wt-CRACM1 (black trace, same data as in FIG. 2A) or D101/112A mutant. Currents were normalized to unity at 120 s (I/I₁₂₀ₛ). Cells expressing the D110/112A mutant were superfused with nominally Ca²⁺-free external solution in the presence (130 mM, n=13) or absence of Na⁺ (TEA⁺ substitution, n=5). Perfusion time is indicated by the black bar. Currents were analyzed as in panel A. (C) Average I/V relationships of CRAC currents extracted from representative HEK293 cells shown in panels A and B. Data represent average leak-subtracted currents evoked by 50 ms voltage ramps from −100 to +150 mV and normalized to cell capacitance (pF). Traces show wt-CRACM1-expressing cells (black trace, n=10; scaled by 1.7 to fit inward currents size of D110/112A mutant) at the end of application of a Na⁺-free solution containing 10 mM Ca²⁺ (180 s) and D110/112A mutants extracted before (at 120 s, blue trace, n=11) or during application of nominally Ca²⁺-free solution containing normal Na⁺ (red trace, n=11). (D) Normalized average time course (I/I₁₂₀ₛ) of IP₃-induced (20 μM) currents produced by the D110/112A mutant in cells superfused with nominally Ca²⁺-free solution containing Na⁺ (red line, same data as in panel B), K⁺ (black circles, n=12) or Cs⁺ (blue circles, n=9). Application time is indicated by the black bar. Currents were analyzed as in panel A. (E) Normalized average time course (I/I₁₂₀ₛ) of IP₃-induced (20 μM) currents produced by wt-CRACM1 (black circles, n=8) or D110/112A mutant (red circles, n=8). Cells were superfused with nominally divalent-free external solution supplemented with 10 μM Ca²⁺ as indicated by the black bar. Currents were analyzed as in panel A. (F) Anomalous mole fraction effect of wt-CRACM1 (black circles, n=5-14) or D110/112A mutant (red circles, n=5-8). Current sizes measured at different Ca²⁺ concentrations were set in relation to current amplitudes obtained with 10 mM Ca²⁺, averaged and plotted against increasing extracellular Ca²⁺ concentrations. (G) Normalized average time course (I/I₁₂₀ₛ) of IP₃-induced (20 μM) currents produced by wt-CRACM1 in cells superfused with an external solution where 10 mM Ca²⁺ was equimolarly substituted with Ba²⁺ (black circles, n=9) or Sr²⁺ (blue circles, n=7) in the absence of Na⁺ (replaced by TEA⁺ to avoid Na⁺ current contamination). Currents were analyzed as in panel A. (H) Normalized average time course (I/I₁₂₀ₛ) of IP₃-induced (20 μM) currents produced by the D110/112A mutant in cells superfused with an external solution where 10 mM Ca²⁺ was substituted equimolarly with Ba²⁺ (black circles, n=7) or Sr²⁺ (blue circles, n=7) in the absence of Na⁺ (replaced by TEA⁺ to avoid Na⁺ current contamination). Currents were analyzed as in panel A. (I) Permeation profile of wt-CRACM1 (black, n=5-12) or D110/112A mutant (red, n=5-14). Currents at −80 mV were assessed at the end of an external application exchange (180 s), set in relation to currents before application (120 s), averaged and plotted as rest current in percent (%). Data were sorted by application condition (10 mM Ca²⁺, 10 mM Ba²⁺, 10 mM Sr²⁺, 130 mM Na⁺, 130 mM K⁺, 130 mM Cs⁺). Monovalent conductances were assessed in nominally Ca²⁺-free solutions in the presence of standard Mg²⁺ concentrations (2 mM). Data represent the summary of panels A through H.

### DETAILED DESCRIPTION OF THE INVENTION

Functional CRACM is required for CRAC channel activity. The invention relates, in part, to methods useful in identifying molecules that bind to CRACM polypeptides, that modulate CRAC ion channel activity by interaction with CRACM, and that alter expression of CRAC polypeptides within cells

CRACM1 is expressed in Drosophila and human. It is believed that CRACM1 is expressed in immune cells. Accordingly, agents that modulate CRAC channel activity via interaction with CRACM1 protein or disruption of CRACM1 expression can be used to modulate inflammatory processes, allergic reactions and auto-immune diseases.

CRACM2 is expressed in Drosophila and has no known ortholog in humans. Agents which disrupt the CRAC channel activity of CRACM2 or which inhibit expression of CRACM2 can be used as pesticides.

As described herein, the term “CRACM” refers to a family of modulators of calcium release activity Ca⁺² (CRAC) channels. CRACM polypeptides are defined by their amino acid sequence, the nucleic acids which encode them, and their properties.

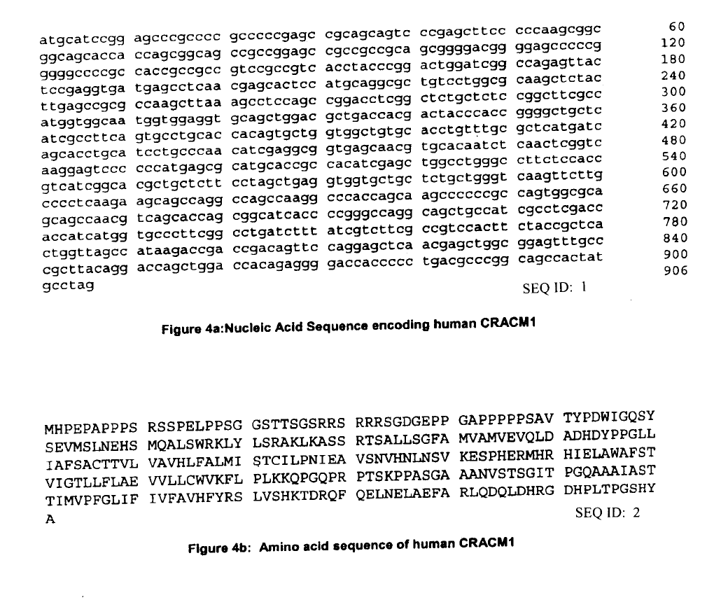

The sequence for human CRACM1 polypeptide is disclosed herein in FIG. 4B. The sequence for Drosophila CRACM1 and CRACM2 can be found on line at (1) Drosophila CRACM1 (olf186-F); http://flybase.bio.indiana.edu/.bin/asksrs.html?%5Blibs %3D %7BFBgn %20 PFgn %7D-all %3AFBgn0041585%5D and (2) Drosophila CRACM2 (dpr3): http://flybase.bio.indiana.edu/.bin/asksrs.html?%5Blibs %3D %7BFBgn %20 PFgn %7D-all %3AFBgn0053516%5D.

The term “CRACM sequence” specifically encompasses naturally-occurring truncated or secreted forms (e.g., an extracellular domain sequence or an amino-terminal fragment), naturally-occurring variant forms (e.g., alternatively spliced forms) and naturally-occurring allelic variants.

The CRACM polypeptide that may be used in the methods of the invention or for other purposes includes polypeptides having at least about 80% amino acid sequence identity, more preferably at least about 85% amino acid sequence identity, even more preferably at least about 90% amino acid sequence identity, and even more preferably at least about 95%, 97%, 98% or 99% sequence identity with the amino acid sequence of SEQ ID NO: 2, or fragments thereof. Such CRACM polypeptides include, for instance, polypeptides wherein one or more amino acid residues are substituted and/or deleted, at the N- or C-terminus, as well as within one or more internal domains. Those skilled in the art will appreciate that amino acid changes may alter post-translational processes of the CRACM polypeptide variant, such as changing the number or position of glycosylation sites or altering the membrane anchoring characteristics. All CRACM polypeptides, however, exhibit one or more of the novel properties of the CRACM polypeptides as defined herein.

“Percent (%) amino acid sequence identity” with respect to the CRACM polypeptide sequences identified herein is defined as the percentage of amino acid residues in a candidate sequence that are identical with the amino acid residues of SEQ ID NO: 2, after aligning the sequences and introducing gaps, if necessary, to achieve the maximum percent sequence identity, and not considering any conservative substitutions as part of the sequence identity. The % identity values may be generated by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a further embodiment, the % identity values used herein are generated using a PILEUP algorithm. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

In yet another embodiment, CRACM polypeptides from humans or from other organisms may be identified and isolated using oligonucleotide probes or degenerate polymerase chain reaction (PCR) primer sequences with an appropriate genomic or cDNA library. As will be appreciated by those in the art, the unique CRACM nucleic acids having nucleotide sequences of SEQ ID NO: 1 or portions thereof, are particularly useful as a probe or PCR primer sequence. As is generally known in the art, preferred PCR primers are from about 15 to about 35 nucleotides in length, with from about 20 to about 30 being preferred, and may contain inosine as needed. The conditions for the PCR reaction are well known in the art.

In a preferred embodiment, CRACM is a “recombinant protein” or “recombinant polypeptide” which is made using recombinant techniques, i.e. through the expression of a recombinant CRACM nucleic acid. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, with at least about 90% being more preferred and at least about 95% being particularly preferred. The definition includes the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or of amino acid substitutions, additions and deletions, as discussed below.

As used herein, “CRACM nucleic acids” or their grammatical equivalents, refer to nucleic acids that encode CRACM polypeptides. The CRACM nucleic acids exhibit sequence homology to CRACM1 and CRACM2 where homology is determined by comparing sequences or by hybridization assays.

A CRACM nucleic acid encoding a CRACM polypeptide is homologous to the DNA sequence forth in FIG. 4A. Such CRACM nucleic acids are preferably greater than about 75% homologous, more preferably greater than about 80%, more preferably greater than about 85% and most preferably greater than 90% homologous. In some embodiments the homology will be as high as about 93%, 95%, 97%, 98% or 99%. Homology in this context means sequence similarity or identity, with identity being preferred. A preferred comparison for homology purposes is to compare the sequence containing sequencing differences to the known CRACM sequence. This homology will be determined using standard techniques known in the art, including, but not limited to, the local homology algorithm of Smith & Waterman, Adv. Appl Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

In a preferred embodiment, the % identity values used herein are generated using a PILEUP algorithm. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

In preferred embodiment, a BLAST algorithm is used. BLAST is described in Altschul et al., J. Mol. Blol. 215:403-410, (1990) and Karlin et al., PNAS USA 90:5873-5787 (1993). A particularly useful BLAST program is the WU-BLAST-2, obtained from Altschul et al., Methods in Enzymology, 266:460-480 (1996); http://blast.wustl/edu/blast/README.html. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a preferred embodiment, “percent (%) nucleic acid sequence identity” is defined as the percentage of nucleotide residues in a candidate sequence that are identical with the CRACM nucleotide residue sequences. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleosides than those of CRACM1 or CRACM2, it is understood that the percentage of homology will be determined based on the number of homologous nucleosides in relation to the total number of nucleosides. Thus, for example, homology of sequences shorter than those of the sequences identified herein and as discussed below, will be determined by using the number of nucleosides in the shorter sequence.

As described above, the CRACM nucleic acids can also be defined by homology as determined through hybridization studies. Hybridization is measured under low stringency conditions, more preferably under moderate stringency conditions, and most preferably, under high stringency conditions. The proteins encoded by such homologous nucleic acids exhibit at least one of the novel CRACM polypeptide properties defined herein. Thus, for example, nucleic acids which hybridize under high stringency to a nucleic acid having the sequence set forth as SEQ ID NO: 1 and their complements, are considered CRACM nucleic acid sequences providing they encode a protein having a CRACM property.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional examples of stringency of hybridization reactions, see Ausubel et al., Current Protocols in Molecular Biology, Wiley Interscience Publishers, (1995), hereby incorporated by reference in its entirety.

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like. Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

In another embodiment, less stringent hybridization conditions are used; for example, moderate or low stringency conditions may be used, as are known in the art. For additional details regarding stringency of hybridization reactions, see Ausubel et al., Current Protocols in Molecular Biology, Wiley Interscience Publishers, (1995).

The CRACM nucleic acids, as defined herein, may be single stranded or double stranded, as specified, or contain portions of both double stranded or single stranded sequence. As will be appreciated by those in the art, the depiction of a single strand also defines the sequence of the other strand; thus the sequences described herein also include the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

The CRACM nucleic acids, as defined herein, are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purposes of the invention. Homologs and alleles of the CRACM nucleic acid molecules are included in the definition.

CRACM sequences can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs such as ALIGN, DNAstar, BLAST, BLAST2 and INHERIT which employ various algorithms to measure homology, as has been previously described.

In another embodiment, the CRACM nucleic acids, as defined herein, are useful in a variety of applications, including diagnostic applications, which will detect naturally occurring CRACM nucleic acids, as well as screening applications; for example, biochips comprising nucleic acid probes to the CRACM nucleic acids sequences can be generated.

In another embodiment, the CRACM nucleic acid sequence is a cDNA fragment of a larger gene, i.e. it is a nucleic acid segment. “Genes” in this context include coding regions, non-coding regions, and mixtures of coding and non-coding regions. Accordingly, as will be appreciated by those in the art, using the sequences provided herein, additional sequences of CRACM genes can be obtained, using techniques well known in the art for cloning either longer sequences or the full length sequences; see Maniatis et al., and Ausubel, et al., supra, hereby expressly incorporated by reference.

Once the CRACM nucleic acid, as described above, is identified, it can be cloned and, if necessary, its constituent parts recombined to form the entire CRACM gene. Once isolated from its natural source, e.g., contained within a plasmid or other vector or excised therefrom as a linear nucleic acid segment, the recombinant CRACM nucleic acid can be further-used as a probe to identify and isolate other CRACM nucleic acids, from other multicellular eukaryotic organisms, for example additional coding regions.

In another embodiment, the CRACM nucleic acid (e.g., cDNA or genomic DNA), as described above, encoding the CRACM polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

A host cell comprising such a vector is also provided. By way of example, the host cells may be mammalian host cell lines which include Chinese hamster ovary (CHO), COS cells, cells isolated from human bone marrow, human spleen or kidney cells, cells isolated from human cardiac tissue, human pancreatic cells, and human leukocyte and monocyte cells. More specific examples of host cells include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR (CHO, Urlaub and Chasin, Proc. Natl. Acad. Sci. USA, 77:4216 (1980)); human pancreatic β-cells; mouse sertoli cells (TM4, Mather, Biol. Reprod., 23:243-251 (1980)); human lung cells (W138, ATCC CCL 75); human liver cells (Hep G2, HB 8065); and mouse mammary tumor cells (MMT 060562, ATCC CCL51). The selection of the appropriate host cell is deemed to be within the skill in the art. In the preferred embodiment, HEK-293 cells are used as host cells. A process for producing CRACM polypeptides is further provided and comprises culturing host cells under conditions suitable for expression of the CRACM polypeptide and recovering the CRACM polypeptide from the cell culture.

In another embodiment, expression and cloning vectors are used which usually contain a promoter, either constitutive or inducible, that is operably linked to the CRACM-encoding nucleic acid sequence to direct mRNA synthesis. Promoters recognized by a variety of potential host cells are well known. The transcription of a CRACM DNA encoding vector in mammalian host cells is preferably controlled by an inducible promoter, for example, by promoters obtained from heterologous mammalian promoters, e.g., the actin promoter or an immunoglobulin promoter, and from heat-shock promoters. Examples of inducible promoters which can be practiced in the invention include the hsp 70 promoter, used in either single or binary systems and induced by heat shock; the metallothionein promoter, induced by either copper or cadmium (Bonneton et al., FEBS Lett. 1996 380(1-2): 33-38); the Drosophila opsin promoter, induced by Drosophila retinoids (Picking, et al., Experimental Eye Research. 1997 65(5): 717-27); and the tetracycline-inducible full CMV promoter. Of all the promoters identified, the tetracycline-inducible full CMV promoter is the most preferred. Examples of constitutive promoters include the GAL4 enhancer trap lines in which expression is controlled by specific promoters and enhancers or by local position effects; and the transactivator-responsive promoter, derived from E. coli, which may be either constitutive or induced, depending on the type of promoter it is operably linked to.

Transcription of a DNA encoding the CRACM by higher eukaryotes may be increased by inserting an enhancer sequence into the vector. Enhancers are cis-acting elements of DNA, usually about from 10 to 300 bp, which act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the CRACM coding sequence, but is preferably located at a site 5′ from the promoter.

Modulation of CRACM

The methods of the invention utilize CRACM polypeptides or nucleic acids which encode CRACM polypeptides for identifying candidate bioactive agents which bind to CRACM, which modulate the activity of CRAC ion channels, or which alter the expression of CRACM within cells.

A preferred aspect of the invention provides for a method for screening for a candidate bioactive agent capable of modulating the ion channel activity of a CRACM polypeptide. In one embodiment, such a method includes the steps of providing a cell expressing the CRACM polypeptide. The cell is contacted with the candidate bioactive agent and the ion channel activity of the CRACM polypeptide is determined both before and after contact between the cell and the candidate bioactive agent. An alteration in ion channel activity of the CRACM polypeptide indicates that the candidate bioactive agent is capable of modulating the activity of the CRACM polypeptide.

One embodiment of the invention provides for a method of screening for a candidate bioactive agent capable of binding to CRACM. In a preferred embodiment for binding assays, either CRACM or the candidate bioactive agent is labeled with, for example, a fluorescent, a chemiluminescent, a chemical, or a radioactive signal, to provide a means of detecting the binding of the candidate agent to CRACM. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound CRACM. As known in the art, unbound labeled streptavidin is removed prior to analysis. Alternatively, CRACM can be immobilized or covalently attached to a surface and contacted with a labeled candidate bioactive agent. Alternatively, a library of candidate bioactive agents can be immobilized or covalently attached to a biochip and contacted with a labeled CRACM. Procedures that may also be used employ biochips and are well known in the art.

The term “candidate bioactive agent” as used herein describes any molecule which binds to CRACM, modulates the activity of a CRACM, or alters the expression of CRACM within cells. A molecule, as described herein, can be an oligopeptide, small organic molecule, polysaccharide, polynucleotide, or multivalent cation etc. Generally a plurality of assay mixtures is run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are multivalent cations or organic molecules, or small organic compounds having a molecular weight of more than 100 and less than about 2,500 Daltons (D). Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Candidate agents may be bioactive agents that are known to bind to ion channel proteins, to modulate the activity of ion channel proteins, or to alter the expression of ion channel proteins within cells. Candidate agents may also be bioactive agents that were not previously known to bind to ion channel proteins, to modulate the activity of ion channel proteins, or alter the expression of ion channel proteins within cells.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of multicellular eucaryotic proteins may be made for screening in the methods of the invention. Particularly preferred in this embodiment are libraries of multicellular eukaryotic proteins, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids.

As described above generally for proteins, nucleic acid candidate bioactive agents may be naturally occurring nucleic acids, random nucleic acids, or “biased” random nucleic acids. For example, digests of prokaryotic or eucaryotic genomes may be used as is outlined above for proteins.

In a preferred embodiment, the candidate bioactive agents are organic chemical moieties, a wide variety of which are available in the literature.

Modulation of CRACM Expression

In a preferred embodiment, anti-sense RNAs and DNAs can be used as therapeutic agents for blocking the expression of certain CRACM genes in vivo. It has already been shown that short antisense oligonucleotides can be imported into cells where they act as inhibitors, despite their low intracellular concentrations caused by their restricted uptake by the cell membrane. (Zamecnik et al., (1986), Proc. Natl. Acad. Sci. USA 83:4143-4146). The anti-sense oligonucleotides can be modified to enhance their uptake, e.g. by substituting their negatively charged phosphodiester groups by uncharged groups. In a preferred embodiment, CRACM anti-sense RNAs and DNAs can be used to prevent CRACM gene transcription into mRNAs, to inhibit translation of CRACM mRNAs into proteins, and to block activities of preexisting CRACM proteins.

Down regulation of the CRACM gene or inhibition of CRACM protein activity reduces the immune response in vertebrates. Bioactive agents such as the ones described herein are useful in the treatment of inflammatory diseases, conditions associated with diseases, or disorders, such as autoimmune disease or graft versus host diseases, or other related autoimmune disorders, wherein the decreased or reduced immune response results in an improved condition of the vertebrate (i.e., the disease condition associated with the disease, or disorder is prevented, eliminated or diminished). Bioactive agents may also be used to reduce allergic reactions.

Another embodiment provides for screening for candidate bioactive agents which modulate expression levels of CRACM within cells. Candidate agents can be used which wholly suppress the expression of CRACM within cells, thereby altering the cellular phenotype. In a further preferred embodiment, candidate agents can be used which enhance the expression of CRACM within cells, thereby altering the cellular phenotype. Examples of these candidate agents include antisense cDNAs and DNAs, regulatory binding proteins and/or nucleic acids, as well as any of the other candidate bioactive agents herein described which modulate transcription or translation of nucleic acids encoding CRACM.

Modulation of Cation Permeability of CRAC Channels

Another embodiment provides for methods of screening for candidate bioactive agents that modulate the Ca⁺² permeability of the CRAC channels. Modulation of the Ca⁺² permeability of the CRAC channel can, for example, be determined by measuring the inward and outward currents in whole cell patch clamp assays or single-channel membrane patch assays in the presence and absence of the candidate bioactive agent. In an alternative embodiment, the modulation of monovalent cation activity is monitored as a function of monovalent cation currents and/or membrane-potential of a cell comprising a CRAC channel. For example, the modulation of membrane potential is detected with the use of a membrane potential-sensitive probe. In a preferred embodiment, the membrane potential sensitive probe is a fluorescent probe such as bis-(1,3-dibutylbarbituric acid)trimethine oxonol (DiBAC4(3)) (Handbook of Fluorescent Probes and Research Chemicals, 9th ed. Molecular Probes, incorporated herein by reference). The use of a fluorescent membrane potential-sensitive probe allows rapid detection of change in membrane potential by monitoring change in fluorescence with the use of such methods as fluorescence microscopy, flow cytometry and fluorescence spectroscopy, including use of high through-put screening methods utilizing fluorescence detection (Alvarez-Barrientos, et al., “Applications of Flow Cytometry to Clinical Microbiology”, Clinical Microbiology Reviews, 13(2): 167-195, (2000)).

Modulation of the cationic permeability of the CRAC channel by a candidate agent can be determined by contacting a cell that expresses CRACM with a divalent cation indicator which reacts with the cation to generate a signal. The intracellular levels of the divalent cation are measured by detecting the indicator signal in the presence and absence of a candidate bioactive agent. Preferred cations enable Ca⁺² Ba⁺², Sr⁺² and Mn⁺². A preferred cation is Ca⁺² although Mn⁺² can be used and detected by its ability to quench fura-2 fluorescence. Another embodiment provides for comparing the intracellular divalent cation levels in cells that express CRAC and CRACM with cells that do not express CRACM in the presence and absence of a candidate bioactive agent.

The levels of intracellular Ca²⁺ levels are detectable using indicators specific for Ca². Indicators that are specific for Ca²⁺ include fura-2, indo-1, rhod-2, fura-4F, fura-5F, fura-6F and fura-FF, fluo-3, fluo-4, Oregon Green 488 BAPTA, Calcium Green, X-rhod-1 and fura-red (Handbook of Fluorescent Probes and Research Chemicals, 9th ed. Molecular Probes).

In a preferred embodiment, both the levels of intracellular Ca²⁺ or other divalent cation and the change in membrane potential are measured simultaneously. In this embodiment a Ca²⁺ specific indicator is used to detect levels of Ca²⁺ and a membrane potential sensitive probe is used to detect changes in the membrane potential. The Ca²⁺ indicator and the membrane potential sensitive probe are chosen such that the signals from the indictors and probes are capable of being detected simultaneously. For example, both the indicator and probe have a fluorescent signal but the excitation and/or emission spectrum of each indicator is distinct, such that the signal from each indicator can be detected at the same time.

CRAC channels are also permeable to monovalent (e.g., such as Na⁺). Accordingly, the modulation of CRAC channel activity by agents that interact with CRACM can be measured using monovalent ions.

As used herein, a monovalent cation indicator is a molecule that is readily permeable to a cell membrane or otherwise amenable to transport into a cell e.g., via liposomes, etc., and upon entering a cell, exhibits a fluorescence signal, or other detectable signal, that is either enhanced or quenched upon contact with a monovalent cation. Examples of monovalent cation indicators useful in the invention are set out in Haugland, R. P. Handbook of Fluorescent Probes and Research Chemicals., 9th ed. Molecular Probes, Inc Eugene, Oreg., (2001) incorporated herein by reference in its entirety.

CRAC channel must be activated by depletion of intracellular Ca²⁺ stores. This can be achieved by, e.g., calcium ionophore, any receptor agonist that produces inositol 1,4,5-trisphosphate (IP3), a suitable Ca²⁺ chelator such as BAPTA, the Ca2+ pump inhibitors thapsigargin or any other SERCA pump inhibitor (e.g., thapsigargin).

In a preferred embodiment of the invention, the CRAC channel is activated by a calcium ionophore. A calcium ionophore is a small hydrophobic molecule that dissolves in lipid bilayer membranes and increases permeability to calcium. Examples of calcium ionophores include ionomycin, calcimycin A23187, and 4-bromocalcimycin A23187 (Sigma-Aldrich catalog 2004/2005, incorporated herein by reference).

In a preferred embodiment, the ion permeability of CRAC channel is measured in intact cells, preferably HEK-293 cells, which are transformed with a vector comprising nucleic acid encoding CRACM and an inducible promoter operably linked thereto. After inducement of the promoter, the CRACM polypeptides are produced. Endogenous levels of intracellular ions are measured prior to inducement and then compared to the levels of intracellular ions measured subsequent to inducement.

Antibodies to CRACM Polypeptides

In still another embodiment, the invention provides antibodies which specifically bind to unique epitopes on the CRACM polypeptide, e.g., unique epitopes of the protein. Such antibodies can be assayed not only for binding to CRACM but also for their ability to modulate CRACM modulators of CRAC channels.

The anti-CRACM antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the CRACM polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The anti-CRACM polypeptide antibodies may further comprise monoclonal antibodies. Such monoclonal antibodies in addition to binding a CRACM polypeptide can also be identified as a bioactive candidate agent that modulates CRACM channel monovalent cation permeability. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the CRACM polypeptide or a fusion protein thereof. Generally, either peripheral blood lymphocytes (“PBLs”) are used if cells of human origin are desired, or spleen cells, kidney cells, or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against a CRACM polypeptide. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The anti-CRACM polypeptide antibodies may further comprise monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The anti-CRACM polypeptide antibodies may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol, 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by the introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The anti-CRACM polypeptide antibodies may further comprise heteroconjugate antibodies. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a further embodiment, the anti-CRACM polypeptide antibodies may have various utilities. For example, anti-CRACM polypeptide antibodies may be used in diagnostic assays for CRACM polypeptides, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Further, CRACM antibodies may be used in the methods of the invention to screen for their ability to modulate the permeability of CRAC channels to monovalent cations.

CRAC Channels and Disease

A number of diseases, including but not limited to immunodeficiency disease, neurological disease, and cardiovascular disease, are associated with mutations in CRAC channels. For example, a genetic defect has been described in which mutations in a key component of CRAC channels result in T lymphocyte malfunction and Severe Combined Immunodeficiency Disease (SCID). (Partiseti et al., J. Biol. Chem. (1994) 269: 32327-35; Feske et al., Nature (2006) 441:179-85). A powerful tool in the study, diagnosis and treatment of these diseases and other CRAC related diseases is the ability to identify (1) the CRAC channel homologs which underlie the Icrac activity in these disease states and (2) agents that modulate such CRAC channels.

The following examples are provided to illustrate the compositions and methods and of the present invention, but not to limit the claimed invention.

### EXAMPLES

### Example 1

### Genome Screen for Identifying the Gene Encoding the CRAC Channel

In order to identify the gene encoding the CRAC channel or other proteins involved in its regulation, a high-throughput, genome-wide RNA interference (RNAi) screen was performed in Drosophila S2R+ cells. The effect of knockdown of the ˜23,000 genes was tested by performing a kinetic [Ca²⁺]i assay in 384-well microplates using an automated Fluorometric Imaging Plate Reader (FLIPR, Molecular Devices) where changes in [Ca²⁺]i were measured in response to the commonly used SERCA inhibitor thapsigargin.

S2R+ cells were dispensed into the dsRNA (0.25 μg/well) containing 384-well plates, in 10 μl of serum-free Schneider's medium (Invitrogen) and incubated for 40 min. After 40 min, cells were topped with 30 μl of 10% serum containing Schneider's medium and incubated for 3 days. On day 3, cells were loaded with a fluorescent Ca²⁺ indicator Fluo-4-AM in Drosophila saline for 1 hr, washed and re-suspended in Ca²⁺-free Drosophila saline containing 0.1 mM EGTA. Each well was first imaged to determine the baseline fluorescence for 1 min. The cells were then stimulated with 2 μM thapsigargin and the resulting Ca²⁺ release due to emptying of ER stores was measured for 5 min. The buffer was then supplemented with 2 mM CaCl₂ and the resulting calcium influx was recorded for another 5 min.

All 63 plates contained dsRNA against stim1 and thread as positive controls, and dsRNA against GFP and Rho1 as negative controls. The entire library was screened in duplicate. To calculate the inhibition of Ca²⁺ influx caused by each of the different dsRNAs, the inhibition seen with positive control stim1 dsRNA was set as 100 and the inhibition seen with the negative control was set as 0. The percent inhibition seen with the remaining 380 genes on each plate were then calculated with respect to controls. A total of 27 genes that reproducibly inhibited calcium influx were evaluated further in a secondary screen using single-cell patch-clamp assays.

Patch-clamp experiments were performed in the tight-seal whole-cell configuration at 21-25° C. High-resolution current recordings were acquired using the EPC-9 (HEKA). Voltage ramps of 50 ms duration spanning a range of −100 to +100 mV were delivered from a holding potential of 0 mV at a rate of 0.5 Hz over a period of 100-300 sec. All voltages were corrected for a liquid junction potential of 10 mV. Currents were filtered at 2.9 kHz and digitized at 100 μs intervals. Capacitive currents were determined and corrected before each voltage ramp. Extracting the current amplitude at −80 mV from individual ramp current records assessed the low-resolution temporal development of both currents. Where applicable, statistical errors of averaged data are given as means ±S.E.M. with n determinations. Standard external solutions were as follows (in mM): 120 NaCl, 2.8 KCl, 10 CsCl, 2 MgCl₂, 10 CaCl₂, 10 HEPES, pH 7.2 with NaOH, 300 mOsm. Standard internal solutions were as follows (in mM): 120 Cs-glutamate, 8 NaCl, 10 Cs-BAPTA, 4 CaCl₂, 3 MgCl₂, 10 HEPES, 0.02 IP₃, pH 7.2 with CsOH, 300 mOsm. For some experiments [Ca²⁺]i was buffered to zero by 10 mM Cs-BAPTA. For passive-depletion experiments, the internal solution was supplemented with Cs-BAPTA in the absence of IP₃ and calcium. In some cells, 10 μM ionomycin was applied for 3 s using a wide-mouth glass pipette.

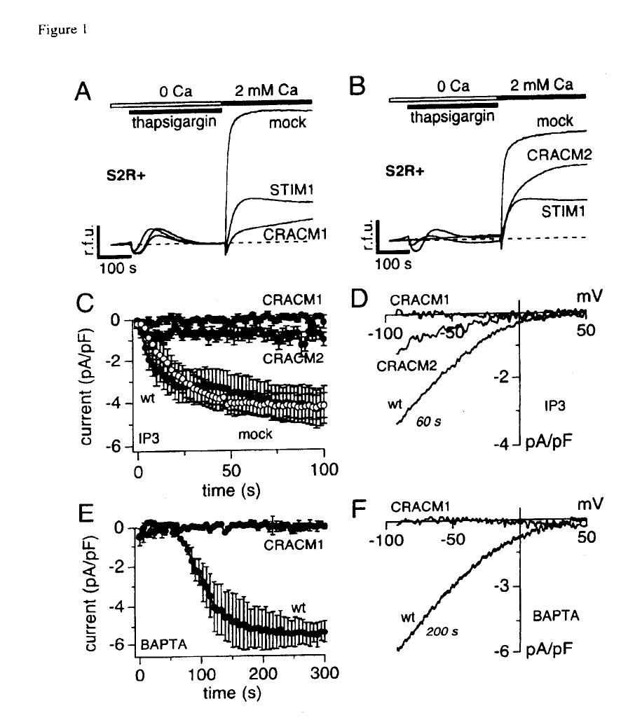

From the secondary patch-clamp screen, 2 novel genes were identified that are essential for CRAC channel function, CRACM1 (encoded by olf-186F in Drosophila and FLJ14466 in human) and CRACM2 (encoded by dpr3 in Drosophila, no human ortholog). FIGS. 1A and 1B show the real-time [Ca²⁺]i imaging data from the wells corresponding to these two genes in the primary screen. The inhibition in calcium influx mediated by CRACM1 and CRACM2 dsRNA is shown in comparison to the negative control Rho1 and positive control stim1. FIGS. 1C and 1D show the time course of inositol 1,4,5-trisphosphate (IP₃)-mediated CRAC current development (assessed by normalized current amplitudes at −80 mV) and the characteristic I/V relationships in Drosophila Kc cells, respectively. Both untreated control wild-type (wt) as well as mock-treated cells responded to IP₃-mediated store depletion by activating an inwardly rectifying Ca²⁺ current typical of I_{CRAC}, which is also present in Drosophila. In contrast, CRAC currents were essentially abolished in cells treated with dsRNA for CRACM1 and CRACM2. In some of the experiments on CRACM1, we also applied ionomycin (10 μM) extracellularly on top of the 20 μM IP₃ included in the patch pipette to ensure complete store depletion, but this also failed to induce I_{CRAC} (n=4, data not shown). As in the active store depletion protocols via IP₃ and ionomycin described above, CRAC currents were also absent when inducing passive store depletion by the Ca²⁺ chelator BAPTA (FIGS. 1E and F).

Since unlike CRACM2, CRACM1 has a human ortholog in gene FLJ14466, we decided to characterize this protein and wanted to confirm that the function of this gene is conserved across species and is involved in store-operated Ca²⁺ entry. To test this, we used siRNA-mediated silencing of human CRACM1 in human embryonic kidney cells (HEK293) and human T cells (Jurkat). Two CRACM1-specific siRNA sequences and one control scrambled sequence were selected and cloned into a retroviral vector, pSUPER.retro (Oligoengine). The siRNA-infected cells were selected using puromycin and used for Ca²⁺ imaging and electrophysiological analyses.

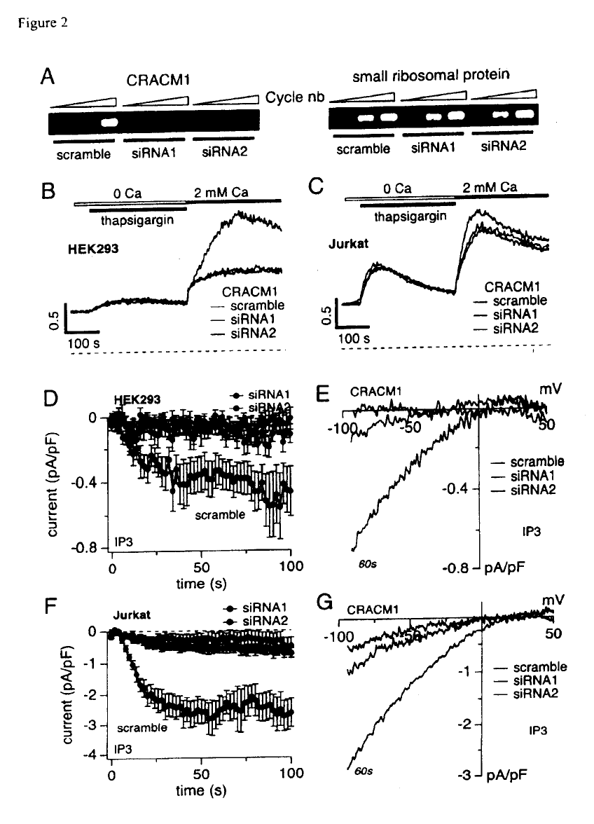

The selective knockdown of CRACM1 message was confirmed by semi-quantitative RT-PCR analysis (FIG. 2A). FIG. 2B illustrates siRNA-mediated inhibition of Ca²⁺ influx in response to thapsigargin-induced store depletion in HEK293 cells. Both of the CRACM1-specific siRNA sequences showed a 60-70% inhibition of calcium influx in response to thapsigargin-induced store depletion in HEK293 cells (FIG. 2B), without affecting the calcium release transient. FIGS. 2D and 2E illustrate the patch-clamp recordings obtained from siRNA-treated HEK293 cells in response to intracellular IP₃ perfusion, demonstrating a nearly complete inhibition of CRAC currents. In Jurkat cells, siRNA-mediated inhibition of the Ca²⁺ signal was close to 20% (FIG. 2C) and not as dramatic as in the HEK293 cells. However, I_{CRAC} in Jurkat cells was effectively reduced by both siRNA sequences (FIGS. 2F and 2G).

### Example 2

### Overexpression of CRACM1 in Cell Lines

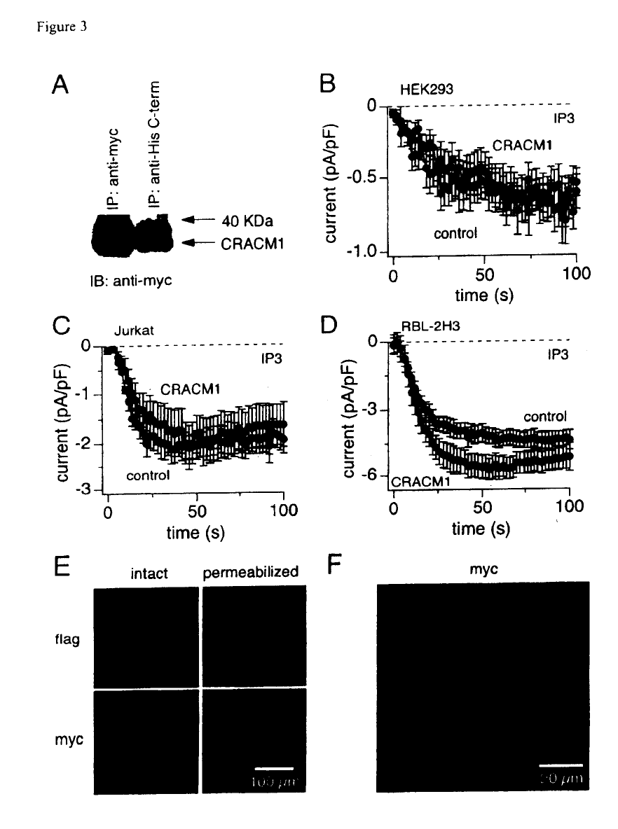

The full length human CRACM1 was cloned in frame with the C-terminal myc-His tag in a pcDNA/4TO/myc-His plasmid (Invitrogen). The full-length gene was re-amplified along with the C-terminal myc-His tag and subcloned into MIGW green fluorescent protein (GFP) retrovirus for overexpression in different cell lines. HEK293, Jurkat, and RBL-2H3 cells were infected with the CRACM1+GFP expressing retrovirus and overexpression of the protein was confirmed in HEK293 cells by IP followed by Western blot using anti-myc tag antibody (FIG. 3A). Overexpression of the CRACM1 protein did not affect the thapsigargin-induced calcium influx in HEK293 cells (data not shown). Similarly, no significant increase in CRAC current amplitudes above control levels was detected in either HEK293 (FIG. 3B) or Jurkat cells (FIG. 3C) and only a slight increase in RBL cells (FIG. 3D). These data demonstrate that CRACM1, while necessary for CRAC activation, does not in and of itself generate significantly larger CRAC currents.

### Example 3

### Localization of CRACM1

CRACM1 is a transmembrane protein involved in store-operated Ca²⁺ entry we wanted to know whether it localized to the ER (like STIM1) or to the plasma membrane. To address this question CRACM1 was tagged on either end and the constructs were transfected into HEK293 cells. After 24 hours, immunofluorescence confocal analysis revealed no staining in intact cells expressing either construct, showing that both tags are intracellular. After permeabilizing the cells, both constructs were clearly detected by the fluorescent antibody and showed predominant peripheral staining of the plasma membrane (FIGS. 3E and 3F). These data fit well with the proposed structure of CRACM1, which contains four predicted transmembrane domains, with both ends facing the cytosol.

In summary, the results from the experiment demonstrate that CRACM1 is essential for store-operated Ca²⁺ influx via CRAC channels. Although overexpression of CRACM1 does not alter the magnitude of CRAC currents, the plasma membrane localization of this protein and the presence of multiple transmembrane domains point towards a more direct role for CRACM1 in store-operated calcium influx. Based on our results, but with no intention of limiting the instant invention to these mechanisms, a number of possible functions can be envisioned for CRAGM1. First, CRACM1 could function as the CRAC channel itself. In this scenario, the unaltered CRAC currents in CRACM1 overexpressing cells might be due to a limiting factor upstream of CRAC channel activation (e.g., STIM1). Second, CRACM1 could be a crucial subunit of a multimeric channel complex, in which case the other subunit(s) could become the limiting factor(s) and prevent CRACM1 overexpression to yield a larger CRAC current. Finally, CRACM1 might not be an integral molecular component of the CRAC channel itself, but rather function as a plasma membrane acceptor or docking protein, possibly for STIM1 or some other as yet unidentified component of the signaling machinery that ultimately leads to CRAC channel activation and store-operated Ca²⁺ entry.

### Example 4

### CRACM1 associates with itself to form the CRAC Channel Complex

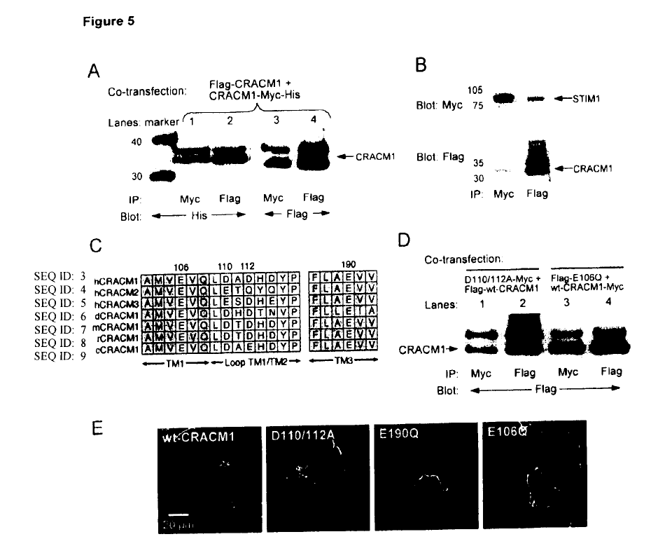

Since many ion channels multimerize to form a functional ion pore, we tested CRACM1's propensity to multimerize by co-overexpressing two differently tagged versions of the protein in HEK293 cells and performing reciprocal co-immunoprecipitation experiments followed by immunoblotting with the relevant anti-tag antibodies. FIG. 5 illustrates that each tagged version of CRACM1 co-immunoprecipitates with the other, indicating that CRACM1 indeed multimerizes with itself. Since STIM1 moves to the plasma membrane following store depletion, it might interact with CRACM1. We tested this using differently tagged CRAGM1 and STIM1 co-overexpressed in HEK293 cells and subjected to reciprocal immunoprecipitation followed by immunoblotting with the relevant anti-tag antibodies. As shown in FIG. 5B, both proteins co-immunoprecipitated, suggesting that they bind to each other.

We analyzed the primary sequence of CRACM1 and identified glutamate residues E106 in TM1 and E190 in TM3, both of which are highly conserved for CRACM1 and its homologs CRACM2/CRACM3 (Orai2/Orai3) as well as across several species (see FIG. 5C). In addition, the first extracellular loop, linking TM1 and TM2 domains, contains several negatively charged aspartate residues (D110, D112 and D114) that could potentially serve as a Ca²⁺ binding site. We constructed several CRACM1 mutants in which we modified these residues to test for their possible involvement in forming the pore of the CRAC channel and conferring the high specificity for Ca²⁺. Co-immunoprecipitation confirmed that these mutant proteins retain the capacity to multimerize (FIG. 5D) and confocal microscopy revealed proper targeting to the plasma membrane (FIG. 5E). We then over-expressed these mutant proteins in HEK293 cells that stably over-express STIM1 and analyzed them electrophysiologically by whole-cell patch-clamp recordings in which we induced CRAC currents by IP₃-mediated Ca²⁺ store depletion.

### Example 5

### Transmembrane Domains 1 and 3 of CRACM1 form the Ca²⁺-Selective ion Channel Pore

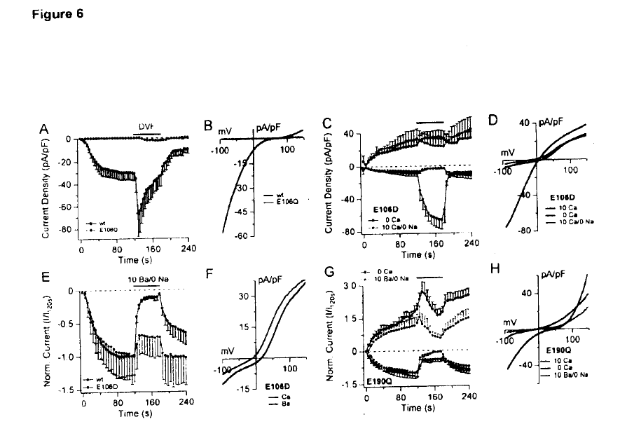

A point mutant of CRACM1 was generated in which the glutamate in TM1 at position 106 was changed to a glutamine residue (E106Q). When transfected into STIM1-overexpressing HEK293 cells, this mutant inhibited thapsigargin-induced Ca²⁺ influx in fura-2 fluorescence measurements (data not shown) and patch-clamp recordings confirmed that this mutant not only failed to produce large CRAC currents as did the wt-CRACM1 (FIGS. 6, A and B), but caused a complete suppression of the small endogenous CRAC currents (˜0.5 pA/pF) typically seen in STIM1 over-expressing cells or untransfected HEK293 cells. Even exposure to divalent-free solution, which in wt-CRACM1 generates large monovalent currents, failed to produce sizeable inward currents (FIG. 6A). Since the mutation did not affect the capacity of CRACM1 to multimerize (FIG. 5D) or its transport to the plasma membrane (FIG. 5E), the E106Q mutant acts as a dominant negative protein that can form normal CRACM1 complexes and even co-assemble with endogenous CRACM1, but is not able to provide a pore that would allow permeation of either Ca²⁺ or Na⁺ ions.

A charge-conserving mutation was generated by converting the glutamate into an aspartate residue (E106D). This mutant exhibited membrane currents that activated similarly as wt-CRACM1 after IP₃-mediated store depletion, but were smaller on average (−8±1 pA/pF, n=12 vs. −30±6 pA/pF, n=14; cf. FIGS. 6, A and C). The selectivity of these mutated CRACM1 channels also differed markedly from wt-CRACM1, converting the typically inwardly rectifying current-voltage relationship into outwardly rectifying and shifting its reversal potential from far positive voltages toward 0 mV (cf. FIGS. 6 B and D). The prominent outward current was flowing through CRAC channels, which developed with exactly the same time course as the inward current and is presumably carried by the major intracellular cation Cs⁺. Upon removal of extracellular Ca²⁺, the current reversed to inward rectification due to a massive increase of inward current and a slight increase in outward current. It should be noted that these effects were obtained by a simple removal of Ca²⁺ while maintaining the presence of 2 mM Mg²⁺, which normally prevents any monovalent inward or outward currents through wt-CRACM1 channels. The large increase in inward current upon removal of Ca²⁺ suggests that the channel still conducts Ca²⁺ ions inwardly when Ca²⁺ ions are present and precludes massive Na⁺ flux. We confirmed this by experiments in which we maintained extracellular Ca²⁺ at 10 mM and replaced extracellular Na⁺ by non-permeant TEA⁺. This caused a reduction in inward current by ˜50% (FIGS. 6, C and D), where the remaining inward current is carried by Ca²⁺ ions and the outward current by the predominant intracellular Cs⁺ ions.

Additional ion-substitution experiments confirmed that the modified selectivity of this mutant is not limited to monovalent cations, but also affects the relative permeability of Ba²⁺ ions. FIGS. 6E and 6F illustrate that the equimolar substitution of Ca²⁺ by Ba²⁺ causes only a small decrease in inward current, which is in marked contrast to the wt-CRACM1 channel, where the same ion substitution reduces inward currents by ˜90%. Thus, the E106D mutant has a significantly increased Ba²⁺ permeation compared to wt. The E106 residue is thus a crucial structural element that confers the CRAC channel's high Ca²⁺ selectivity und unequivocally demonstrates that CRACM1 indeed represents the pore-forming subunit of the CRAC channel.

Sequence analysis reveals another acidic and negatively charged residue in TM3 (E190) that is equally well conserved across CRACM proteins. We constructed a mutant in which we replaced this glutamate by a glutamine residue (E190Q mutation). When expressed into STIM1-expressing HEK293 cells, we found that this mutant activated normally following IP₃-induced store depletion and generated inward currents that were primarily carried by Ca²⁺, since removal of extracellular Ca²⁺ (while maintaining 2 mM Mg²⁺) reduced inward by about 70% (FIG. 6G). The remaining Na⁺ current is larger than in wt-CRACM1, suggesting reduced selectivity for Ca²⁺ over Na⁺. However, in marked contrast to the E106D mutant, inward currents did not increase. Interestingly, the outward current through the E190Q mutant was more prominent and linear than that of the E106D mutant (FIG. 6H), suggesting that monovalent outward permeation of Cs⁺ is significantly enhanced in this mutant.

Ba²⁺ permeability of the E190Q mutant was investigated, which is very low in wt-CRACM1, but significantly increased in the E106D mutant. Substitution of Ca²⁺ by Ba²⁺ resulted in almost complete abolition of inward current with only 5% of inward current remaining under Ba²⁺ (FIG. 6G). The E190Q mutant thus retains high Ca²⁺ selectivity over Ba²⁺ similar to wt-CRACM1.

### Example 6

### The First Extracellular Loop of CRACM1 Contributes to the Pore Selectivity

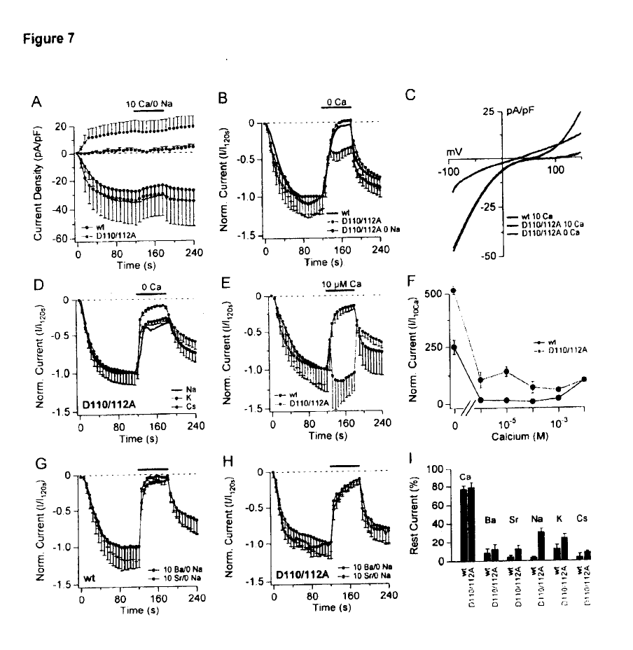

Adjacent to the critical E106 residue, there are three closely spaced aspartate residues (D110/112/114) in the first extracellular loop of CRACM1, which may participate in coordinating the binding of Ca²⁺ at the outer mouth of the channel. A double mutant was generated in this region by changing the most conserved negatively charged aspartate residues at positions 110 and 112 into alanines (D110/112A mutation). The predominant plasma membrane localization of this mutant as well as its multimerization potential were comparable to wt-CRACM1 (FIGS. 5 D and E). The CRAC currents generated by the D110/112A mutant activated with a similar time course as those produced by the wt channel (FIG. 7A). The inward currents of both constructs at −80 mV also were quite similar, however, the mutant showed a distinctive and much larger outward current at +130 mV than the wt channel. The current-voltage relationships of the wt and mutant constructs illustrate these features in more detail (FIG. 7C). Thus, at negative voltages, both constructs exhibit similar inwardly rectifying currents, whereas at voltages more positive than +80 mV the D110/112A mutant passes a significantly larger amount of outward current. FIG. 3A also demonstrates that the inward currents of both wt and mutant channels remained largely unaffected when removing extracellular Na⁺ by replacing it with TEA⁺ and maintaining 10 mM Ca²⁺ as the only charge carrier. This suggests that both channel constructs retain high selectivity for Ca²⁺ over Na⁺ influx when 10 mM Ca²⁺ is present extracellularly.

However, since outward movement of monovalent cations was enhanced in the D110/112A mutant, monovalent inward currents were measured at low extracellular Ca²⁺ by ion substitution experiments in which extracellular Ca²⁺ was removed. When removing Ca²⁺, while retaining 130 mM Na⁺ and 2 mM Mg²⁺, the wt CRAC current is essentially abolished (FIG. 7B), demonstrating that the remaining Mg²⁺ completely prevents monovalent Na⁺ permeation. In contrast, the inhibition of the inward current by the D110/112A mutant is not as complete, suggesting that the absence of Ca²⁺ allows for more Na⁺ permeation than in wt. The remaining Na⁺ inward current was then blocked completely when replacing extracellular Na⁺ by TEA⁺ (FIG. 7B). Additional experiments revealed that the D110/112A mutant also allows limited permeation of K⁺ ions, but negligible permeation of Cs⁺ in the inward direction (FIG. 7D). The aspartate residues in the loop between TM domains 1 and 2 thus contribute to the selectivity profile of CRACM1 channels, presumably by coordinating Ca²⁺ binding to the outer mouth of the channel and thereby contributing to the discrimination of Ca²⁺ ions against monovalent cations, although the instant invention is not limited to this mechanism.

Based on the above results, one would expect the D110/112A mutant to modify the interplay of divalent and monovalent permeation, which in the wt CRAC channel manifests itself in a dose-response curve for extracellular Ca²⁺ with a characteristic anomalous fraction behavior (FIG. 7F). Thus, in the complete absence of extracellular divalent ions (nominally divalent-free solution +10 mM EDTA), CRAC channels allow significant Na⁺ permeation. However, exposing cells to just nominally divalent free solutions without EDTA (free Ca²⁺ and Mg²⁺ estimated at ˜1 μM) or adding 10 μM Ca²⁺, virtually eliminates inward currents in wt-CRACM1 channels (FIG. 7E). As Ca²⁺ is increased into the millimolar range, CRAC currents increase again due to selective Ca²⁺ permeation. The inhibitory effect of low Ca²⁺ concentrations on Na⁺ permeation could be mediated by the binding of Ca²⁺ to the aspartate residues in the first extracellular loop. Indeed, the D110/112A mutant produces significant inward currents even when 10 μM Ca²⁺ is present extracellularly (FIG. 7E), changing the anomalous mole fraction behavior of CRAC channels (FIG. 7F). At higher concentrations of Ca²⁺, the current again behaves similar to the wt and becomes Ca²⁺, selective.

The selectivity of this mutant among divalent cations was measured. When replacing extracellular Ca²⁺ by equimolar Ba²⁺ or Sr⁺, wt-CRACM1 currents are significantly smaller than those carried by Ca²⁺, amounting to <10% (FIG. 7G). FIG. 7H shows that the D110/112A mutant produced only marginally increased Ba²⁺ and Sr²⁺ currents, indicating that the mutant largely retains relative selectivity for divalent cations. The bar graph in FIG. 7I summarizes the relative magnitude of inward current carried by divalent and monovalent cations in wt and D110/112A CRACM1 channels, demonstrating similar divalent permeation, but significantly increased permeation of Na⁺ in particular.

Taken together, the results of the present study demonstrate that the CRACM1 protein forms multimeric ion channel complexes in the plasma membrane, where they can be activated following Ca²⁺ store depletion, presumably by interacting with STIM1. The channel pore of CRACM1 is highly selective for Ca²⁺ ions owing to the presence of critical glutamate residues in TM1 and TM3 (E106 and E190) as well as aspartate residues (D110 and D112) within a Ca²⁺-binding motif located in the extracellular loop that connects TM1 and TM2. Mutations of either of these critical residues alter the CRAC channel selectivity by enhancing monovalent cation permeation relative to Ca²⁺, providing unambiguous evidence that CRACM1 harbors the CRAC channel pore.

What is claimed is:

1. A method for screening for a candidate bioactive agent capable of modulating the activity of a CRACM polypeptide, the method comprising:
a) providing a cell, wherein said cell expresses the CRACM polypeptide;
b) contacting the cell with the candidate bioactive agent; and
c) measuring the expression or ion channel activity of the CRACM polypeptide, wherein an alteration in the expression or ion channel activity of the CRACM polypeptide as compared to the expression or ion channel activity of the CRACM polypeptide in the absence of said candidate bioactive agent indicates that the candidate bioactive agent is capable of modulating the activity of the CRACM polypeptide.

2. The method of claim 1, wherein said ion channel activity comprises store operated calcium entry.

3. The method of claim 1 wherein said CRACM polypeptide is a CRACM1 polypeptide.

4. The method of claim 1 wherein the CRACM polypeptide is a CRACM2 polypeptide.

5. A method for screening for a candidate bioactive agent capable of modulating divalent cationic permeability of a cell comprising:
a) contacting a cell expressing CRACM with a candidate agent; and
b) detecting whether the candidate agent modulates the divalent cationic permeability of the cell.

6. The method of claim 5 wherein the divalent cationic permeability of the cell is increased by the contacting with the candidate agent.

7. The method of claim 5 wherein the divalent cationic permeability of the cell is decreased by the contacting with the candidate agent.

8. The method of claim 5 wherein the divalent cation is selected from the group consisting of Ca⁺², Ba⁺², Sr⁺² and Mn⁺².

9. A method for screening for a bioactive agent capable of binding to a CRACM polypeptide comprising:
a) providing a recombinant cell comprising a recombinant nucleic acid expressing CRACM polypeptide;
b) contacting the recombinant cell with a candidate agent; and
c) detecting modulation of Ca⁺² permeability of the cell;
wherein modulation of Ca⁺² permeability indicates that the bioactive agent is capable of binding to the CRACM polypeptide.

10. The method of claim 1, wherein the Ca⁺² permeability is increased by the candidate agent.

11. The method of claim 11, wherein the Ca⁺² permeability is decreased by the candidate agent.

12. The method of claim 9, wherein said CRACM is CRACM1.

13. The method of claim 9, wherein said CRACM is CRACM2.

14. A method for screening for a candidate bioactive agent capable of binding to a CRACM polypeptide, the method comprising:
a) contacting a CRACM polypeptide with the candidate agent; and
b) determining the binding of the candidate agent to the CRACM polypeptide.

15. The method of claim 14, wherein a library of two or more of the candidate agents are contacted with the CRACM polypeptide.

16. The method of claim 14, wherein said CRACM polypeptide is a CRACM1 polypeptide.

17. The method of claim 14, wherein the CRACM polypeptide is a CRACM2 polypeptide.

18. A method for inhibiting CRAC activity comprising contacting at least one cell with an agent that inhibits CRACM expression.

19. A method for inhibiting CRAC activity comprising contacting at least one cell with an agent that inhibits the CRAC activity of a CRACM polypeptide.

20. The method of claim 18 or 19 wherein CRACM is CRACM1 or CRACM2.

21. The method of claim 20, wherein said agent is an antisense CRACM1 nucleic acid.

22. The method of claim 20, wherein said agent is an antisense CRACM2 nucleic acid.

23. The method of claim 20, wherein said agent is an anti-CRAC1 antibody.

24. The method of claim 20, wherein said agent is an anti-CRAC2 antibody.
